(19) United States
(12) Patent Application Publication
(10) Pub. No.: US 2023/0167481 A1
(43) Pub. Date: Jun. 1, 2023

(54) Improved Fluidic Device

(71) Applicants: UNIVERSITEIT ANTWERPEN, Antwerpen (BE); VRIJE UNIVERSITEIT BRUSSEL, Brussel (BE)

(72) Inventors: Tatevik CHALYAN, Brussel (BE); Wendy MEULEBROECK, Brussel (BE); Kurt ROCHLITZ, Brussel (BE); Stijn VERWULGEN, Antwerpen (BE); Sander VAN GOETHEM, Antwerpen (BE); Chris BAELUS, Antwerpen (BE); Muriel DE BOECK, Antwerpen (BE); Herman GOOSSENS, Antwerpen (BE); Heidi OTTEVAERE, Brussel (BE); Hugo THIENPONT, Brussel (BE); Pieter MOONS, Antwerpen (BE)

(21) Appl. No.: 17/922,210

(22) PCT Filed: May 4, 2021
(86) PCT No.: PCT/EP2021/061718

§ 371 Date: Oct. 28, 2022

(30) Foreign Application Priority Data
May 4, 2020 (EP) 20172725.2

(51) Int. Cl. C12Q 1/20; B01L 3/00; C12M 3/06

(52) U.S. Cl. CPC C12Q 1/20; B01L 3/502715; C12M 23/16; B01L 2300/0681; B01L 2300/168; B01L 2200/16; B01L 2300/0861; B01L 2300/0654

(57) ABSTRACT

The invention relates in a first aspect to an improved fluidic device for determining a property of a microbe. The device comprising a bottom layer comprising a plurality of light-transmissive wells; and a top layer comprising an injection opening and a fluid distribution system. Each of said plurality of distribution channels has: essentially the same channel length between the inlet end and the outlet end; and essentially the same channel volume. The invention relates in a second aspect to a use of the device for determining a susceptibility of a microbe, preferably a bacterium, to an antimicrobial drug.

### FIELD OF THE INVENTION

The present invention relates to a fluidic device adapted for optical analysis of its contents. In particular, the device is adapted for determining a susceptibility of a bacterial culture to an antimicrobial drug.

### BACKGROUND

There remains a need in the state of the art to simplify production of fluidic devices. There also remains a need for improving fluidic device designs for determining a minimum inhibitory concentration of an antimicrobial drug and/or for determining a property related to heteroresistivity of a microbe.

### SUMMARY OF THE INVENTION

The present invention and embodiments thereof serve to provide a solution to one or more of above-mentioned disadvantages.

The fluidic device according to the present invention is advantageous for fast and accurate optical analysis of its contents, for instance the determination of the susceptibility of a bacteria culture to a microbial drug.

The composition of the fluidic device allows equal distribution of the to be tested bacterial culture between different wells. The filling of said wells is controlled in time and volume, e. g. wells are simultaneously filled with an essentially equal interaction volume, resulting in an accurate minimum inhibitory concentration determination, compared to current standard practices known in the art (e.g. Etest). The fluidic device of the invention is however not limited for use in determination of minimum inhibitory concentration, and can easily be used for other optical analysis processes which make use of fluidic volumes of the substance to be analyzed.

In used well systems (such as a microplate) each well has to be filled manually, which is labor intensive and prone to mistakes. This problem could be solved by using a liquid handling robot; however, this would result in a more expensive and bigger setup, which is not desirable. By having an injection opening and a fluid distribution system, the person conducting or preparing the test only has to place the liquid in the injection opening, eliminating volume variations between the different wells and eliminating potential interactions which are commonly present when using a chip comprising multiple wells, e.g. due to mutual contamination caused by liquids flowing from one well to another, and thus allowing for more controlled growth conditions, confined to small, standardized and portable dimensions, and thus allowing for accurate susceptibility testing. Ideally, the wells should thus have the same volume and the same optical path length. Nonetheless, embodiments can be envisioned of the invention where the wells do not necessarily have the same path length but do, for example, possess the same available volume.

Moreover, the construction is beneficial for eliminating gas bubbles present in the fluidic device. Gas bubbles may arise from injecting the fluid into the fluidic device, but even after injection the bacteria may continuously generate other gas bubbles. These bubbles can constrain correct measurements of the bacteria growth, and as a consequence can influence the accuracy of the minimum inhibitory concentration determination.

DE 198 10 499 A1 and US 2011/036862 A1 disclose fluidic chips for analyzing samples, but do not sufficiently overcome the raised issues.

Further advantages, features, and examples of the present invention are disclosed in the detailed description.

### DESCRIPTION OF FIGURES

The following numbering refers to: (60) injection opening; (61) light transmissive well; (62) bifurcating channel; (63) filling channel; (64) waste well; (65) bottom layer; (67) porous membrane; (68) vent opening; (69) drainage channel; (70) hump portion; (73) outer sub-layer; (74) inner sub-layer; (75) waste lead channel; (76) waste opening; (100) outlet end of the fluid distribution system; (101) sealing rubber; (102) inlet channel.

The following description of the figures of specific embodiments of the invention is merely exemplary in nature and is not intended to limit the present teachings, their application or uses. Throughout the drawings, corresponding reference numerals indicate like or corresponding parts and features.

FIGS. 1A and 1B show respectively a top and a perspective view of a fluidic device in accordance with an embodiment of the disclosure.

FIG. 2 shows a perspective view of a fluidic device in accordance with an embodiment of the disclosure.

FIG. 3 shows a cross-sectional view in the lateral direction of an embodiment of a fluidic device in accordance with an embodiment of the disclosure.

FIG. 4 shows a cross-sectional view in the lateral direction of an embodiment of a fluidic device in accordance with an embodiment of the disclosure.

FIG. 5 shows a cross-sectional view in the lateral direction of an embodiment of a fluidic device in accordance with an embodiment of the disclosure.

FIG. 6 shows a cross-sectional view in the lateral direction of an embodiment of a fluidic device in accordance with an embodiment of the disclosure.

FIGS. 7A and 7B show a plot of the optical efficiency of a laser beam under a certain shift (X/Y) with respect to the entrance of the well.

### DETAILED DESCRIPTION OF THE INVENTION

The present invention concerns a fluidic device adapted to be used in the optical analysis of its contents, and in particular adapted to be used for the determination of a susceptibility of a bacterial culture to an antimicrobial drug.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the,” as used herein, refers to both singular and plural referents unless the context clearly dictates otherwise. By way of example, “a compartment” refers to one or more than one compartment.

“About”, “essentially”, “substantially” and other relative terms, as used herein, refer to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of,” as used herein, are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

A first aspect of the invention relates to a multi-layer microfluidic device. The device comprising a bottom layer and a top layer. The bottom layer comprising a plurality of light-transmissive wells. The top layer comprising an injection opening. The top layer further comprising a fluid distribution system. The fluid distribution system comprising a plurality of distribution channels. The injection opening is in fluid connection to each of said plurality of distribution channels at an inlet end of each of said plurality of distribution channels. Each of said plurality of distribution channels is in fluid connection to one of said plurality of light-transmissive wells at an outlet end of each of said plurality of distribution channels. Preferably, each of said plurality of distribution channels has: essentially the same channel length between the inlet end and the outlet end of each of said channels; and essentially the same channel volume.

A second aspect of the invention relates to use of the microfluidic device according to the first aspect of the invention, for determining a susceptibility of a microbe, preferably a bacterium, to an antimicrobial drug.

The present invention relates to a multi-layer microfluidic device and a use thereof.

A person having ordinary skill in the art will appreciate that the device and any use thereof are related to the same invention. In what follows, both aspects of the present invention are therefore treated together.

“Microfluidic device” and “microfluidic chip,” as used herein, should be understood as synonyms, and refer to a term known in the state of the art related to a device comprising one or more channels through which a fluid can flow and wherein at least one channel has a dimension in a magnitude of micrometer, tens of micrometers, hundreds of micrometers or thousands of micrometers.

The multi-layer microfluidic device is suited for determining a property of a microbe. “Microbe” and “microorganism,” as used herein, should be understood as synonyms, and refer to a term known in the state of the art related to a microscopic organism, which may exist in its single-celled form or in a colony of cells. The determined property may or may not relate to a physical and/or chemical property of the microbe. Examples of a physical property include a microbial grow rate, a microbial binding affinity, etc. Examples of a chemical property include an antimicrobial resistivity, a metabolite production rate, etc.

The device is particularly suited for determining a susceptibility of a microbe to an antimicrobial drug. “Microbial drug” or “antimicrobial drug,” as used herein, refers to a general term known in the state of the art related to any agent that kills microorganisms (e.g. bacteria or fungi) or stops their growth. The term may or may not refer to antibiotics or antifungals.

The device is particularly suited for determining a minimum inhibitory concentration (MIC) of an antimicrobial drug, i.e. the concentration at which visible growth of a microbe is prevented. In particular, the device is also suited for determining a property related to heteroresistivity of a microbe. Preferably, a microbe, as used herein, refers to a bacterial culture.

An infection with a heteroresistant organism is difficult or even impossible to detect by standard antibiotic susceptibility culturing methods. The applicant has noticed that by employing a third derivative of a growth curve of a microbe, the minimum inhibitory concentration can be accurately detected through a simple experimental setup. The applicant notes that such setup requires a microbe, a growth medium for said microbe, an antimicrobial drug and a technical setup (e.g. a light source in combination with a photodetector or spectrophotometer system with a microfluidic device) for measuring the microbial growth in time. Compared to other methods employing a technological setup, the above method is beneficial for reducing time and cost of testing the microbial susceptibility. Moreover, the applicant has noticed that by using the above method, the minimum inhibitory concentration for some bacterial cultures can be determined after 1 to 6 hours of bacterial growth.

“Growth medium,” as used herein, refers to any composition suitable for growing a bacterial culture, e.g. blood or broth, wherein blood medium may or may not comprise a mixture of blood and broth. The growth medium can be an enrichment culture medium, a selective culture medium, a differential culture medium or a resuscitation culture medium.

The current state of the art does not provide in a microfluidic device suitable for sufficiently accurate growing a microbe at different inhibitory concentrations and/or for detecting the resulting growth-curves according to the above method. The device according the invention provides for a microfluidic device adapted to be used according to the above method.

A simple embodiment of the device according to the current invention comprises a bottom layer and a top layer. Ideally, the device is substantially planar. The bottom layer comprises a plurality of wells. The top layer comprises an injection opening and a fluid distribution system. The fluid distribution system is in fluid connection to each of said plurality of distribution channels, which are each in turn in fluid connection to one of said plurality of light-transmissive wells. The layered configuration discussed hereabove allows for a gravity-assisted filling of the light-transmissive wells. The layered configuration discussed hereabove also prevents transportation of air to the wells as air bubbles are pushed to the entrance of the distribution system by the liquid. Upon entering the distribution system by the fluid, the filling of the channels is partially gravity-assisted and thereby also prevents backflow of fluid from the wells to the distribution system. The layered configuration furthermore improves gas exchange between the well and the environment. These effects will be further illustrated below. Furthermore, by decoupling the filling layer from the well layer, i.e. microbial growth section, by incorporating these different functions in a two (or more) layer system, the production process of the device is simplified. Moreover, the layers are beneficial for further functionalization of the device, in particular for further functionalization of the channels, wells, etc., as will be illustrated below.

“Fluid connection,” as used herein, refers to a general term known in the state of the art, related to a connection allowing the flow of a fluid or liquid from one compartment to another. Preferably, fluid connections, as disclosed herein, relate to one-way connections. Thereby, the distributed fluid remains in each respective compartment and disadvantageous phenomena, such as backflow, contamination, etc., are prevented. Such one-way functionality can be obtained by using a number of techniques known in the art. Examples include, but are not limited to, the use of capillaries, adhesive properties, hydrophilic characteristics of materials/coatings, hydrophobic characteristics of materials/coatings, etc.

Preferably, the device comprises at least two layers. Such device configuration is advantageous for minimalizing the device dimensions as well as for reducing material cost thereof. Preferably, the device comprises at least three layers, i.e. a top layer comprising two or more sub-layers and a bottom layer. Preferably, the top layer comprises an outer sub-layer and an inner sub-layer.

Preferably, the device is adapted to be used in the optical analysis of its contents, and in particular in the fabrication, fill and finish, lab-operation or determination of a susceptibility of a bacteria culture to an antimicrobial drug. As a consequence, the bottom layer comprises a plurality of light-transmissive wells. Preferably, each of said plurality of light-transmissive wells has one or more of: a substantially similar bottom surface, shape, size, volume, etc. Ideally, each of said plurality of cells is the same. Equal volumes in equally dimensioned wells allow measurements with the same optics, electronics and software. Such configuration is thereby ideal for conducting comparative experiments in a repeatable manner. For example, by providing a bacterial inoculate and a different concentration of a compound in each of said wells. Alternatively, some of the light-transmissive wells may or may not have a deviating shape and/or size.

In order to detect bacterial growth, the wells need to comprise a minimal volume. In order to obtain accurate and reproducible results when illuminating a well, the dimensions of the fluidic device should be matched to the dimensions of the illumination beam. The applicant notes that well dimensions comprising a well length between 0.5 cm and 1.5 cm, a well width between 1 mm and 2 mm and a well depth between 1 mm and 2 mm are especially beneficial for obtaining accurate and reproducible MIC values and/or for heteroresistancy determination while not requiring a large sample volume.

Preferably, the distance between the light-transmissive wells is at least 1 μm, more preferably at least 100 μm, even more preferably at least 200 μm, even more preferably at least 300 μm, even more preferably at least 400 μm, even more preferably at least 500 μm, even more preferably at least 600 μm, even more preferably at least 700 μm, even more preferably at least 800 μm, even more preferably at least 900 μm, even more preferably at least 1 mm, even more preferably at least 2 mm, even more preferably at least 3 mm.

Preferably the distance between the light-transmissive wells is at most 20 cm, more preferably at most 18 cm, even more preferably at most 16 cm, even more preferably at most 12 cm, even more preferably at most 10 cm, even more preferably at most 8 cm, even more preferably at most 6 cm, even more preferably at most 4 cm, even more preferably at most 2 cm, even more preferably at mot 1 cm, even more preferably at most 9 mm, even more preferably at most 8 mm, even more preferably at most 7 mm, even more preferably at most 6 mm, even more preferably at most 5 mm, even more preferably at most 4 mm.

Most preferably, the distance between the light-transmissive wells is about 3.4 mm. The applicant notes that a distance between the light-transmissive wells between 3 mm and 4 mm is beneficial for obtaining accurate and reproducible MIC values and/or for heteroresistancy determination, especially when combined with the mentioned well dimensions, while minimizing the dimensions of the device.

Preferably, the device is dimensioned to be handheld, e.g. appr. a length between 5 and 20 cm, a width between 1 and 5 cm and a thickness between 0.2 and 2 cm, to be manipulated by lab-operator in a user-friendly way. In particular, it is desirable to keep at least one dimension, preferably the thickness, of the device relatively low to create a compact, easily stackable (both in use as in storage) device. The above dimensions are such that a multitude of chips e.g. 10 to 500 can be inserted in an incubator with integrated MIC value assessment.

Each of the plurality of wells comprises a well length, a well width and a well depth.

Preferably, the well length is at least 1 μm, more preferably at least 100 μm, even more preferably at least 200 μm, even more preferably at least 300 μm, even more preferably at least 400 μm, even more preferably at least 500 μm, even more preferably at least 600 μm, even more preferably at least 700 μm, even more preferably at least 800 μm, even more preferably at least 900 μm, even more preferably at least 1 mm, even more preferably at least 2 mm, even more preferably at least 3 mm, even more preferably at least 4 mm, even more preferably at least 5 mm, even more preferably at least 6 mm, even more preferably at least 7 mm, even more preferably at least 8 mm, even more preferably at least 9 mm, even more preferably at least 1 cm. Preferably, the well length is at most 20 cm, more preferably at most 18 cm, even more preferably at most 16 cm, even more preferably at most 12 cm, even more preferably at most 10 cm, even more preferably at most 8 cm, even more preferably at most 6 cm, even more preferably at most 4 cm, even more preferably at most 2 cm, even more preferably at most 1 cm. Preferably, the well length is between 0.8 cm and 1.2 cm, more preferably the well length is 1 cm.

Preferably, the well width is at least 1 μm, more preferably at least 100 μm, even more preferably at least 200 μm, even more preferably at least 300 μm, even more preferably at least 400 μm, even more preferably at least 500 μm, even more preferably at least 600 μm, even more preferably at least 700 μm, even more preferably at least 800 μm, even more preferably at least 900 μm, even more preferably at least 1 mm, even more preferably at least 1.1 mm, even more preferably at least 1.2 mm, even more preferably at least 1.3 mm, even more preferably at least 1.4 mm, even more preferably at least 1.5 mm. Preferably, the well width is at most 1 cm, more preferably at most 9 mm, even more preferably at most 8 mm, even more preferably at most 7 mm, even more preferably at most 6 mm, even more preferably at most 5 mm, even more preferably at most 4 mm, even more preferably at most 3 mm, even more preferably at most 2 mm, even more preferably at most 1.5 mm.

Preferably, the well depth is at least 1 μm, more preferably at least 100 μm, even more preferably at least 200 μm, even more preferably at least 300 μm, even more preferably at least 400 μm, even more preferably at least 500 μm, even more preferably at least 600 μm, even more preferably at least 700 μm, even more preferably at least 800 μm, even more preferably at least 900 μm, even more preferably at least 1 mm, even more preferably at least 1.1 mm, even more preferably at least 1.2 mm, even more preferably at least 1.3 mm, even more preferably at least 1.4 mm, even more preferably at least 1.5 mm. Preferably, the well depth is at most 1 cm, more preferably at most 9 mm, even more preferably at most 8 mm, even more preferably at most 7 mm, even more preferably at most 6 mm, even more preferably at most 5 mm, even more preferably at most 4 mm, even more preferably at most 3 mm, even more preferably at most 2 mm, even more preferably at most 1.5 mm.

Preferably, a susceptibility of a bacteria culture to a drug is tested by sending light through the interaction volume of the light-transmissive wells and thereafter measuring a light intensity and/or a light intensity variation. Such light intensity variation may, for example, be determined using a first and/or higher derivate of the light transmitted through the light-transmissive wells as is for example discussed hereabove. Preferably, in such set-up, each of said plurality of light-transmissive wells comprises an antimicrobial drug at two or more substantially different concentrations. Ideally, a gradient of different drug concentrations is provided in the plurality of light-transmissive wells. Preferably, the microbial drug is printed on one or more inner surfaces of the wells. Ideally, each well is subsequently inoculated using a fluid comprising a microbial culture. Preferably, the microbial drug will dissolve into the fluid once the well is completely filled with the fluid. The previous disclosure makes sure the tests in the different wells are automatically started at the correct time point. The microbial growth and antimicrobial resistivity of the microbial culture can subsequently be determined by measuring the light intensity and light intensity variations as is for example discussed above.

“Interaction volume,” as used herein, refers to a general term known in the state of the art, preferably related to a volume that interacts with a light source (e.g. with a laser beam or any other kind of light beam). Interaction volume may or may not alternatively refer to the surface of an illumination spot multiplied by the optical path length of the light through a liquid volume.

“Intensity of the transmitted light,” as used herein, refers to a general term known in the state of the art, preferably to be interpreted as an absorbance, whereby the absorbance is preferably determined by the Lambert-Beer law. In general, the person skilled in the art will appreciate the close correlation of a detected/measured intensity of light traversing a medium and the absorbance for the medium that can be deduced from such a detected intensity.

Preferably, the light-transmissive wells are illuminated with a light sent through the length of the light-transmissive wells. Hereby, the length of the light-transmissive wells forms an optical path length. Preferably, the light originates from a laser. Preferably, is the light collimated along the entire optical path length. Preferably, the collimated light is parallel to the direction of the device length. In this case, the light passes through the well parallel to the inner bottom and/or top surface of the well. The inner bottom and/or top surface of the well does not need optical quality, as the light does not interact with the top and/or bottom surface, allowing the use of cheap materials.

Alternatively, the wells can be illuminated at an angle. The light will hereby make an angle between 1° and 89° with the normal of a side wall, through which the well is illuminated. The light will travel through the well by interacting multiple times with the inner bottom and/or top surface (e.g. through reflection). The previous is beneficial for increasing the optical path length, without increasing the well dimensions and as such the sample volume. However, in this case the inner bottom and/or top surface needs optical quality, e.g. a near perfect reflection surface for making sure that the light does not lose intensity just by interacting with an inner top and/or bottom surface, which would affect the accuracy of the measured transmitted light). Alternatively, the inner bottom and/or top surface are provided with a reflective coating. Preferably, the light spot shape is circular.

Preferably, the light spot diameter is between 500 nm and 1 cm, more preferably between 1 μm and 1 cm, even more preferably between 0.5 mm and 5 mm, even more preferably about 1 mm.

Preferably, the light-transmissive wells are illuminated with a power density of between 0.1 mW/mm3 and 5 mW/mm3, more preferably between 0.2 mW/mm3 and 1 mW/mm3, even more preferably with a power density of 0.5 mW/mm3.

Preferably, each well comprises a minimal interaction volume of 5.0 mm³, preferably 7.5 mm³, more preferably 10.0 mm³, even more preferably 15.0 mm³ or even 20.0 mm³.

Preferably, each well comprises a maximal interaction volume 200.0 mm³, preferably 150.0 mm³, more preferably 125.0 mm³, even more preferably 100.0 mm³, or even 80.0 mm³, 70.0 mm³, 60.0 mm³, 50.0 mm³, 40.0 mm³ or 30.0 mm³.

Preferably, at least a portion of a top side of the light-transmissive wells is hydrophilic and/or comprises a hydrophilic coating. Such hydrophilic portion helps pushing the bubbles to a side and is therefore beneficial for the removal of air bubbles in the optical path length of the light-transmissive wells. Alternatively, at least one, preferably at least two, more preferably at least three, even more preferably at least four, even more preferably at least five, inner surfaces of the wells are hydrophilic or comprise a hydrophilic layer.

Preferably, at least a portion of a top side of the light-transmissive wells is hydrophobic and/or comprises a hydrophobic coating. Such hydrophobic portion helps pushing the bubbles to the side and is therefore beneficial for the removal of air bubbles in the optical path length of the light-transmissive wells. Alternatively, at least one, preferably at least two, more preferably at least three, even more preferably at least four, even more preferably at least five, inner surfaces of the wells are hydrophobic or comprise a hydrophobic layer. A hydrophobic portion in a channel impedes capillary flow by aqueous liquids. Consequently, a hydrophobic portion may be configured to form a capillary valve in a channel. Aqueous liquids are not able to pass this valve. However, when pressurized such liquids are able to bypass the capillary valve. The liquid can be pressurized by injection. Such hydrophobic portion may thus prevent spontaneous backflow of liquid from a well to a channel. Preferably, the upper surface of the well is hydrophobic (most preferably with the other surfaces being hydrophilic), as this will increase the chances that any air bubbles present in the well, will ‘stick’ to said hydrophobic surface, thereby collecting the air bubbles at a singular place (or layer in the well). This ensures that the rest of the well is relatively bubble-free, allowing the light to travel through the center of the well unimpeded (i.e., without facing interference from the bubbles). Note that such configurations with one or more hydrophobic surfaces may also present options to offset the light somewhat from a central passage, away from the hydrophobic surfaces, to reduce risk of interference, while still keeping the light beam inside of the well.

Preferably, at least a first portion of a top side of the light-transmissive wells is hydrophilic and/or comprises a hydrophilic coating and at least a second portion of a top side of the light-transmissive wells is hydrophobic and/or comprises a hydrophobic coating. A configuration comprising both hydrophilic and hydrophobic surfaces is beneficial, firstly because the air bubbles will be more easily removed from the fluidic device and secondly because a flow-back of the fluid is prevented, thereby ensuring that the plurality of wells remain filled throughout the duration of an experiment.

A simple embodiment of the device comprises a fluid distribution system comprising a plurality of distribution channels. The injection opening is in fluid connection to each of said plurality of distribution channels at an inlet end of each of said plurality of distribution channels. Each of said plurality of distribution channels is in fluid connection to one of said plurality of light-transmissive wells at an outlet end of each of said plurality of distribution channels.

The device is advantageous for the controlled distribution of fluid, e.g. substantially even distribution volume, substantially simultaneous of a fluid, which may or may not comprise a bacteria concentration, and for simultaneously filling multiple light-transmissive wells with the fluid. Preferably, each of said plurality of distribution channels has essentially the same channel length between the inlet end and the outlet end of each of said channels. Preferably, each of said plurality of distribution channels has essentially the same channel volume, preferably determined between an inlet end and an outlet end. A cross-sectional area distribution from the inlet end to the outlet end along a fluid path will also essentially be the same of each of the plurality of distribution channels. As a consequence, to the above configurations, fluid injected in the injection opening will have substantially identical physical characteristics upon arrival of said injected fluid in each of said plurality of light-transmissive wells. Examples of such physical characteristic include volumetric pressure, volumetric flow rate, mass flow rate, turbulence, etc.

To clarify the above, the injection opening is directly connected to the inlet ends of the distribution channels. This ensures an equal path length for each of the wells, for liquid travelling from the injection opening (i.e. where the fluid is provided to the device) to the outlet end where the distribution channels deliver the fluid to the wells. As mentioned, this has the advantage of ensuring the wells are filled simultaneously and under essentially the same circumstances, giving a more uniform character to the samples in each well. As such, the path length (for a fluid to travel) between the injection opening and the outlet end of the distribution channel is essentially equal for each well.

The applicant notes that identical physical characteristics of the injected fluid are crucial for determining a MIC of an antimicrobial drug for a microbe or a property related to a heteroresistivity of a microbe, using the above method. Said method uses a first and/or higher, preferably a third, derivate of the light transmitted through a light-transmissive wells of a microfluidic device. The derivative of a microbial growth function measures the sensitivity to change of microbial growth in time. As a consequence, in order to obtain sufficiently accurate growth curves, each of said plurality of well has to be inoculated at the same time using the same quantity. The more simultaneous the growth of the microbe in each well is started, the better the representability of the obtained results and the more accurate and reproducible the MIC value determination will be. All the above considerations are especially true for fast-growing bacterial cultures. Furthermore, the applicant also notes that each inoculate is ideally also subjected to a similar amount of shear, turbulence, acceleration, etc. The applicant furthermore also notes that the even distribution of fluid across the different wells is important in order to not waste fluid. Especially for biological samples, whereby the available volume is limited, it is of extreme importance to handle fluid as efficiently as possible.

According to a preferred embodiment, a cross-sectional area of each of said plurality of distribution channels decreases near the outlet end. The applicant notes that by narrowing the cross-sectional area, e.g. by narrowing the circumference, of the channels of the fluid distribution system near the outlet ends, the distribution of fluid to the plurality of light-transmissive wells is improved. According to a preferred embodiment, the distribution channels may comprise a gradually decreasing cross-sectional area near the outlet ends of the fluid distribution system. Alternatively, the decrease of the cross-sectional area of the distribution channels near the outlet ends of the fluid distribution system is abrupt.

In order to comply to the above requirements, each of the plurality of distribution channels is ideally connected to a single injection opening. Alternatively, two or more injection openings are also possible. In order to simultaneously fill the wells in the bottom layer, said injection openings have to be filled simultaneously, for example using pipetting tool, specifically a multi-channel pipetting tool.

In order to prevent leakage of injected fluid, the device is preferably provided with a fitting suitable for receiving an injection tool. Preferably, said fitting is rubber, latex, etc. Preferably, said fitting is provided at and/or in said injection opening.

A microfluidic device complying with the above constraints may or may not have a circular shape or a polygonal shape, wherein the injection opening is provided in a centre portion of the device and wherein each of the plurality of wells is provided further from said centre portion at an equal distance to said centre portion. “Central portion”, “centre portion” or “core portion”, of the plate, as used herein, should preferably be understood as a portion of the device, concentrically, with respect to said device, provided in said device, wherein said portion has a shape substantially similar to a shape of said device and wherein said portion has a surface area that is at least 90% of a surface area of said device, more preferably at least 80% and most preferably at least 70% of a surface area of said device. Preferably, wherein the device is substantially planar.

Preferably, the device has a rectangular shape, as such shape allows for a parallel configuration of the plurality of wells. Such parallel configuration allows for an accurate, easy and quick detection of a growth curve. Furthermore, such shape allows for an improved stacking and storage of the device. As a consequence of a rectangular shape of the device, the channel length and volume constriction and/or a parallel configuration of the plurality of wells, at least some of the distribution channels require one or more bends. In order to limit the number of channels, at least some of the distribution channels require one or more bifurcations.

Preferably, each of said plurality of distribution channels comprises at least one bend. Depending on the number of wells, the dimensions of the device, the dimensions of the channels, the positioning of the injection opening, etc., the number of bends may vary for each channel. Each distribution channel may or may not comprise more than two, such as three, four, five, six, seven, eight, nine, ten or any other natural number of, bends.

Preferably, the device comprises a fluid distribution system comprising a plurality of bifurcating channels. Preferably, each of said plurality of bifurcating channels comprises two or more of the plurality of distribution channels. Each distribution channel is connected to one well. As a consequence, a bifurcating channel comprising for example two distribution channels, comprises one main channel ending in two bifurcations. Each channel originating from one of said two bifurcations is thereby connected to a well.

Preferably, each of said plurality of bifurcating channels comprises at least one, more preferably at least two, bifurcations. Each bifurcating channel may or may not comprise more than two, such as three, four, five, six, seven, eight, nine, ten or any other natural number of, bifurcations.

Preferably, at each bifurcation the bifurcating channel bifurcates in at least two channels. At each bifurcation the bifurcating channel may or may not bifurcate in more than two, such as three, four, five, six or seven, channels.

According to an alternative embodiment, the device comprises shorter and longer bifurcating channels, wherein the shorter bifurcating channels comprise more bends and/or bifurcations compared to the longer bifurcating channels. Such fluid distribution system lay-out also allows for a simultaneous filling of the plurality of light-transmissive wells. Turbulence and shear are however increased by an increasing number of bends and/or bifurcations. Therefore, such configuration is less ideal than the configuration lay-outs discussed hereabove.

The device preferably comprises a waste system. The use of a waste system is beneficial to contain any spillover of the microbe and for ensuring all light-transmissive wells are completely filled with the microbe even if not all of said wells are simultaneously filled. This connection might be a one-way exit valve not to induce contamination from the waste well to the growth and test wells.

According to a preferred embodiment, the device comprises a waste drainage system and a waste well. The waste drainage system comprises a plurality of drainage channels. Preferably, the top layer of the device comprises the waste well. The use of waste wells is beneficial for containing any spillover within the device, such that wells can be filled to a substantially full capacity. Each of said plurality of drainage channels is in fluid connection to one of said plurality of light-transmissive wells at an inlet end of each of said plurality of drainage channels. Each of said plurality of drainage channels is in fluid connection to said waste well at an outlet end of each of said plurality of drainage channels. By providing the waste well in the top layer, backflow, contamination, etc., are prevented. Preferably, the waste drainage system is provided above each light-transmissive well, preferably in the top layer of the device, thus providing an overflow-configuration for containing any spillover of microbes and/or growth medium and for ensuring that all light-transmissive wells are completely filled with a microbial culture even if the plurality of the light-transmissive wells are not simultaneously filled, which may for example occur due to air trapped in one of the distribution channels.

According to a preferred embodiment, the device comprises one or more waste wells. The device may or may not comprise one waste well. The device may or may not comprise an equal number of waste wells to the plurality of bifurcating channels. Preferably, the device comprises an equal number of waste wells to the plurality of light-transmissive wells. As a consequence, cross-contamination between wells is easily prevented. Each light-transmissive is thus preferably in fluid connection with one of said drainage channels.

According to a preferred embodiment, each of the plurality of distribution channels and/or each of said plurality of drainage channels comprises an inlet portion at the inlet end of each channel, an outlet portion at the outlet end of each channel and a transportation portion connecting both said inlet portion and said outlet portion.

Preferably, the transportation portion is substantially provided along an outer surface of said top layer and/or along an outer surface of a sub-layer comprised by said top layer. The outer surface of said top layer and/or said sub-layer comprised by said top layer is connected to an adjacent sub-layer or said bottom layer.

By providing the transportation portion along the outer surface of said top layer or said sub-layer, the production process of the device is simplified. The transportation portion can for example be produced by forming a groove in the outer surface of said top layer or said sub-layer. By connecting the outer surface of said top layer or said sub-layer to an adjacent sub-layer or the bottom layer, the groove is closed off and the transportation portion of a channel is formed.

By providing the transportation portion along the outer surface of said top layer or said sub-layer, the different channel portions can also be further functionalized. For example, by producing the transportation channel using a groove, deposition of a coating on an inner surface of the channels is facilitated. A coating may serve the purpose of adapting light-transmissive properties of certain sections of the inner surface of the channels. Tuning light-transmissive properties of channel sections can be interesting for improving optical read-outs, etc. A coating may also serve the purpose of adapting hydrophilic/hydrophobic properties of certain sections of the inner surface of the channels. Tuning hydrophilic/hydrophobic properties of channel sections can be interesting for controlling the filing of the plurality of light-transmissive wells, the equal distribution of the fluid in the plurality of light-transmissive wells, holding and/or fixating the fluids in the plurality of light-transmissive wells, controlling the removal of gasses (e.g. obtained by bacteria growth) and for ensuring an accurate optic read out. Preferably, a coating is applied in a separate process under highly controlled circumstances (cleanroom, not to induce contamination by other biologicals).

Preferably, the inlet portion should be understood as an inlet channel. A first end of the inlet channel preferably corresponds to the inlet end of the distribution channel. A second end of the inlet channel preferably corresponds to an end in fluid connection to the transportation portion. Preferably, the inlet channel is provided coaxially to the injection opening. The inlet channel may or may not be provided perpendicular to the plane of the layer in which the inlet channel is provided. The inlet channel may or may not be positioned under an angle to the plane of the layer in which the inlet channel is provided. Said angle preferably ranging from 30° to 150°, more preferably ranging from 45° to 135°. Preferably, the inlet channel is provided in the top layer or any sub-layer thereof. More preferably, the inlet channel extends through the layer in which it is provided. Such configuration is beneficial, as the inlet channel may simply be produced by drilling a hole through the layer in which it is provided. As a consequence, the production process of the device is simplified. Furthermore, the inlet portion can thus also be further functionalized, for example by applying a coating as dissed hereabove.

Preferably, the outlet portion should be understood as a filling channel. A first end of the filling channel preferably corresponds to an end in fluid connection to the transportation portion. A second end of the filling channel preferably corresponds to the outlet end of the distribution channel. Preferably, the filling channel is provided perpendicular to the plane of the layer in which the filling channel is provided. As a consequence, the inlet channel and the filling channel are preferably provided parallel to each other. Preferably, the inlet channel is provided in the top layer or any sub-layer thereof. More preferably, the inlet channel extends through the layer in which it is provided. Such configuration is beneficial, as the inlet channel may simply be produced by drilling a hole through the layer in which it is provided. As a consequence, the production process of the device is simplified. Furthermore, the inlet portion can thus also be further functionalized, for example by applying a coating as dissed hereabove.

According to a preferred embodiment, the device comprises a semi-permeable membrane. Preferably, the membrane is adapted for preventing a fluid from passing through said membrane and for allowing air to pass through said membrane. Preferably, membrane is disposed over at least a portion of a top side of each of said plurality of distribution channels and/or at least a portion of a top side of said plurality of drainage channels and/or at least a portion of a top side of said waste well. Preferably, an outer surface of said membrane is provided along an outer surface of said microfluidic device. As a consequence, the membrane is in gaseous connection to an environment.

By providing a semi-permeable membrane, as disclosed hereabove, in gaseous connection to the environment, the device configuration is exceptionally suitable for removing of carbon dioxide from the channels and wells, while providing oxygen to the fluidic device and thus also to the wells. This is beneficial as it stimulates bacterial growth. As a consequence, more accurate MIC value results are obtained, as the better the conditions for growth, the more representative the results are.

According to a preferred embodiment, the membrane is substantially disposed over the outlet portion of each of said plurality of distribution channels and/or each of said plurality of drainage channels. Preferably, the membrane is substantially disposed over the outlet portion, i.e. the filling channel, of each of said plurality of distribution channels. By positioning the porous membrane near the filling channel opening the diffusion length of the gasses (whereby oxygen will diffuse from the atmosphere into the fluidic device and carbon dioxide from the fluidic device to the atmosphere) is shortened. This is beneficial for the bacteria growth. Preferably, the filling channel is provided perpendicular to the plane of the layer in which the filling channel is provided. As a consequence, communication between air and/or carbon present in the well or the environment is more easily exchanged.

By providing a semi-permeable membrane, as disclosed hereabove, in gaseous connection to the environment, the device configuration is exceptionally suitable for removing air bubbles formed in the device. The presence of air bubbles is disadvantageous for the employment of spectrophotometry or optical detection in general. Air bubbles interfere with the passage of light through the wells and can thus result in wrong transmitted light intensity read-outs. In particular, the device is suited for removing air bubbles formed in the distribution channels, drainage channels and the waste well. The presence of air bubbles in a distribution channel may result in a non-simultaneous filling of the light-transmissive well, which may in turn result in inaccurate growth functions. The presence of air bubbles in a drainage channel may result in a hindrance of the drainage from the wells, which may in turn result in overflow or spillage. The presence of air bubbles in a waste well may result in an inadequate utilization of the present volume of the waste well. A portion of a top side of said waste well may or may not be open as to allow overflow of the waste well and thus prevent backflow to the wells. Such configuration is however less desirable as it increases the probability of contamination. The device may or may not comprises an equal number of outlet channels as the number of waste wells, whereby the outlet channels are in fluid or gaseous communication with the environment. Preferably, each drainage channel is fluid connection with one outlet channel. The outlet channels may or may not be located in the top layer.

According to a preferred embodiment, each of said plurality of distribution channels and/or each of said plurality of drainage channels comprises a hump portion. Preferably, said hump portion is provided below the semi-permeable membrane. The hump portion narrows the cross-sectional area of a channel, which in turn increases the volumetric flow rate of a fluid. This results in a suction flow of the fluid and thereby prevents the passage of air bubbles. The presence of the hump in a channel also physically prevents large air bubbles from passing. Furthermore, the hump portion directs the fluid in the channel to a portion of a top side of the channel. Preferably, said portion of the top side of the channel is provided with a membrane in gaseous connection to the environment as is for example discussed hereabove. Such configuration directs bubbles that passed the hump portion to the membrane and thereby enhances the passing of air from said bubbles to the environment. Ideally, the membrane is thus substantially disposed over said hump portion.

Preferably, the hump portion is provided in a transportation portion of a distribution channel and/or a drainage channel. The transportation portion is traditionally provided parallel to the plane of the layer wherein it is provided. As consequence, said portion has the highest chance of air build-up. Ideally, the hump portion should thus be provided in the transportation portion of a distribution channel and/or a drainage channel, preferably at the end of said transportation portion, as to prevent the passing of air to the wells.

Preferably, the hump portion extends along a width of a distribution channel and/or a drainage channel. As a consequence, all fluid passing said hump is directed to a portion of a top side of a channel. Furthermore, such configuration prevents turbulence. Preferably, the hump portion extends along a height of a distribution channel and/or a drainage channel. Taking into account the above constraints regarding the location of the channels, the fluid passing said hump is directed to an above sub-layer or to an upper surface of the top layer.

According to a preferred embodiment, the membrane is adaptable between a flat resting state and a convex filling state. Preferably, the membrane is flat and parallel to the upper surface of the fluidic device, when provided in said flat resting state. Preferably, the membrane has a concave shape with respect to the bottom surface of the fluidic device when provided in the filling state. According to the above configuration, the at least one porous membrane is pushed upwards by the fluid pressure generated during the filling process, allowing the passing of air from said bubbles to the environment via the membrane.

According to a preferred embodiment, the membrane touches the hump portion when said membrane is provided in the flat resting state. Preferably, the membrane allows fluid passage between the hump portion and the membrane when said membrane is provided in the convex filling state. Such configuration provides a synergy between the function of the membrane, i.e. the passing of air from said bubbles to the environment, and a one-way connection between a first and section of a channel. In the case of a distribution or drainage channel as said channel, a one-way connection is provided between the inlet end and the outlet end, thereby preventing backflow, etc.

According to a preferred embodiment, the membrane is disposed over the top layer of said microfluidic device. Such configuration allows for an easy production, as the membrane can simply be attached to the outer surface of the device. Furthermore, such configuration forgoes the need for multiple sub-layers within the top layer of the device.

According to a preferred embodiment, the top layer comprises an outer sub-layer and inner sub-layer. Preferably, the membrane is disposed between said outer sub-layer and said inner sub-layer. As a consequence, to the above configuration, the membrane is held in place and is protected from damages such as the puncturing the membrane. Preferably, said outer sub-layer is provided with a plurality of vent openings disposed over said membrane, in order to provide a gaseous connection between the air entrapped in the device and the environment. The device may or may not comprise an equal number of vents as the number of light-transmissive wells. The device may or may not comprise a number of vents smaller than the number of light-transmissive wells.

Preferably, an end of the vents is located in the top layer. Preferably, the end of the air openings has an angle between 0° and 89° with an upper surface of the top layer.

Preferably, the air openings have a diameter of between 0.001 mm to 1 mm, more preferably between 0.01 mm and 1 mm, even more preferably between 0.05 mm and 1 mm, even more preferably between 0.1 mm and 1 mm, even more preferably between 0.2 mm and 0.9 mm, even more preferably between 0.3 mm and 0.8 mm, even more preferably between 0.4 mm and 0.7 mm, even more preferably between 0.4 mm and 0.6 mm, even more preferably said diameter is 0.5 mm.

Preferably, the membrane is hydrophobic. Hydrophobic membranes are beneficial for ensuring that liquid does not pass through the membrane, which is advantageous for maintaining the bacteria in the fluidic device.

According to a preferred embodiment, the bottom layer of the device comprises one or more materials chosen from the group comprising: glass, silicon, ceramic, elastomers, poly-di-methyl-siloxane, thermoset polyester, thermoplastic polymer, polystyrene, polycarbonate, poly-methyl methacrylate, poly-ethylene glycol diacrylate, perfluorinated compounds, polyurethane, paper, hydrogel or cyclic-olefin copolymer. More preferably, the bottom layer of the device comprises cyclic-olefin copolymer, poly-methyl methacrylate and/or poly-di-methyl-siloxane.

According to a preferred embodiment, the top layer of the device comprises one or more materials chosen from the group comprising: glass, silicon, ceramic, elastomers, poly-di-methyl-siloxane, thermoset polyester, thermoplastic polymer, polystyrene, polycarbonate, poly-methyl methacrylate, poly-ethylene glycol diacrylate, perfluorinated compounds, polyurethane, paper, hydrogel or cyclic-olefin copolymer. More preferably, the top layer of the device comprises cyclic-olefin copolymer, poly-methyl methacrylate and/or poly-di-methyl-siloxane.

According to a preferred embodiment, any sub-layer comprised by the top layer comprises one or more materials chosen from the group comprising: glass, silicon, ceramic, elastomers, poly-di-methyl-siloxane, thermoset polyester, thermoplastic polymer, polystyrene, polycarbonate, poly-methyl methacrylate, poly-ethylene glycol diacrylate, perfluorinated compounds, polyurethane, paper, hydrogel or cyclic-olefin copolymer. More preferably, the top layer of the device comprises cyclic-olefin copolymer, poly-methyl methacrylate and/or poly-di-methyl-siloxane.

According to a preferred embodiment, each of said plurality of light-transmissive wells comprises a first wall portion and a second wall portion. Preferably, said first and second wall portion are provided in a parallel configuration. The above configuration is ideally provided perpendicularly to an optical light path.

According to a preferred embodiment, the first and second wall portion have an RMS surface roughness equal to at most a light wavelength (λ) used during optical analysis of the device's content divided by ten. The amount of scattered light at a surface of a wall portion of a well is directly related to the surface roughness of said wall portion. A high surface roughness of wall sections results in emission light lost by scattering effects. Therefore, light scatter and roughness should be minimized. The inventors noted that light scatter is acceptable, as long as an RMS surface roughness is equal to at most a A used during optical analysis of the device's content divided by ten. Preferably, during optical analysis of the device's content, light is used with a wavelength between 250 and 1600 nm. Accordingly, the first and second wall portion should have an RMS surface roughness of at most 25 and at most 160 nm, respectively.

In a preferred embodiment, the first and second wall portion have a RMS surface roughness of at most 160 nm, and preferably above 25 nm. Other upper and/or lower boundaries for the RMS roughness can be envisioned, such as 30 nm, 35 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm. Note that these are paired to wavelengths that would be used that are at least equal to 10 times the roughness, so respectively at least 300 nm, 350 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 700 nm, 750 nm, 800 nm, 850 nm, 900 nm, 950 nm, 1000 nm, 1050 nm, 1100 nm, 1150 nm, 1200 nm, 1250 nm, 1300 nm, 1350 nm, 1400 nm, 1450 nm, 150 nm, 1550 nm, and at least 1600 nm. For instance, in devices aimed at being used with illumination of 450 nm, the RMS roughness would necessarily be at most 45 nm.

In a specifically preferred embodiment, the RMS roughness is between 40 and 65 nm, which is coupled to a wavelength range between 400 and 650 nm (but which can thus be higher as it would still satisfy the desired ratio), with notably preferred embodiments with a roughness of about 45 nm or 63.5 nm, which are coupled to (minimal) wavelengths of 450 nm and 635 nm respectively.

Specifically, the device is adapted to achieve a certain optical quality in a particular range of wavelengths, thus optimizing the use of the device for determining susceptibilities to microbes, bacteria, and antimicrobial drugs, wherein a ratio of the wavelength over the RMS roughness is achieved of at least 10 (or the surface roughness equal to at most the wavelength divided by 10). Preferably, this is the case in the wavelength range between 250 nm and 1600 nm. The optical quality is governed by the formula S=([2(n−1)πσ]/λ)², with σ being the RMS roughness, lambda the wavelength and n the refractive index. Preferred pairings of roughness of the wall portions and the wavelengths employed in the use can be found in the preceding paragraph, although not limited thereto.

In the second aspect, the invention can thus be further distinguished by having a roughness of at most 160nm, and preferably at least 25 nm. More preferably this is at most 65 nm and at least 40 nm, and even more preferably of about 45 nm or 63.5 nm. Most preferably, this applies to the use of the device under a respective wavelength range between 250 nm and 1600 nm (or higher), between 400 nm and 650 nm (or higher), and of about 450 nm and 635 nm minimally.

“Roughness,” as used herein, relates to the irregularities in the surface texture. Irregularities are the peaks and valleys of a surface. The roughness value may or may not be computed by AA (arithmetic average) and RMS (root-mean-square). The AA method uses the absolute values of the deviations in the averaging procedure, whereas the RMS method utilizes the squared values of the deviations in the averaging process.

An alternative use of the device, is the interpretation of absorption results for a bacterial culture in a blood medium. To accommodate said use, the fluidic device is preferably provided with two or more, preferably two, injection openings, i.e. a first injection opening and a second injection opening. This is beneficial for a correct interpretation of the absorption results for a bacteria culture in a blood medium. The absorption properties of a blood medium may not be constant over time, and as such it is beneficial to simultaneous monitor the absorption properties of the blood medium as well as the bacterial growth. In order to keep blood with and without bacteria separated, two injection openings would ideally be required. Preferably, at least one injection opening is in fluid connection with at least two, preferably at least three bifurcating channels. Preferably, the device further comprises one non-bifurcating channel, whereby the non-bifurcating channel is in fluid connection to one light-transmissive well. Preferably, the first injection opening is in fluid connection with the non-bifurcation channel and the light-transmissive well, whereby the non-bifurcation channel is configured for filling one light-transmissive well. Preferably, the second injection opening is in fluid connection with the bifurcation channels.

The invention is further described by the following non-limiting examples which further illustrate the invention, and are not intended to, nor should they be interpreted to, limit the scope of the invention.

### EXAMPLES

The present invention will now be further exemplified with reference to the following example(s). The present invention is in no way limited to the given examples or to the embodiments presented in the figures.

### Example 1: Different Embodiments of the Fluidic Device

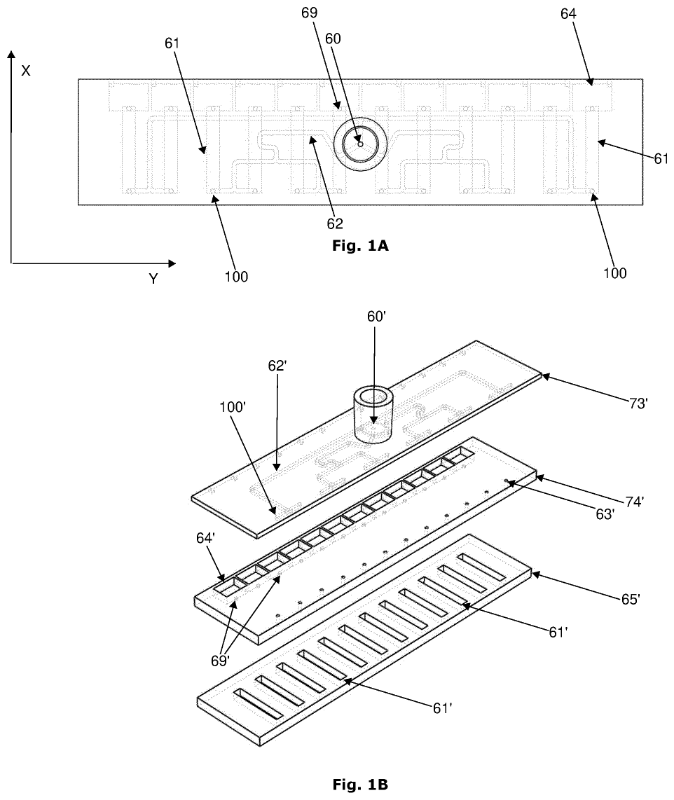

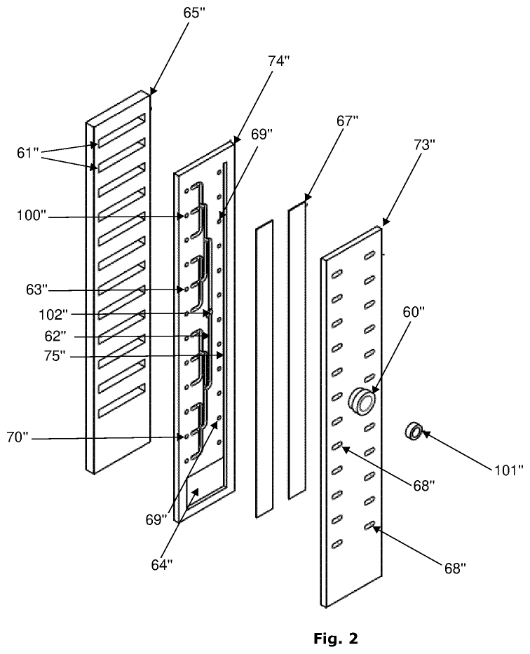

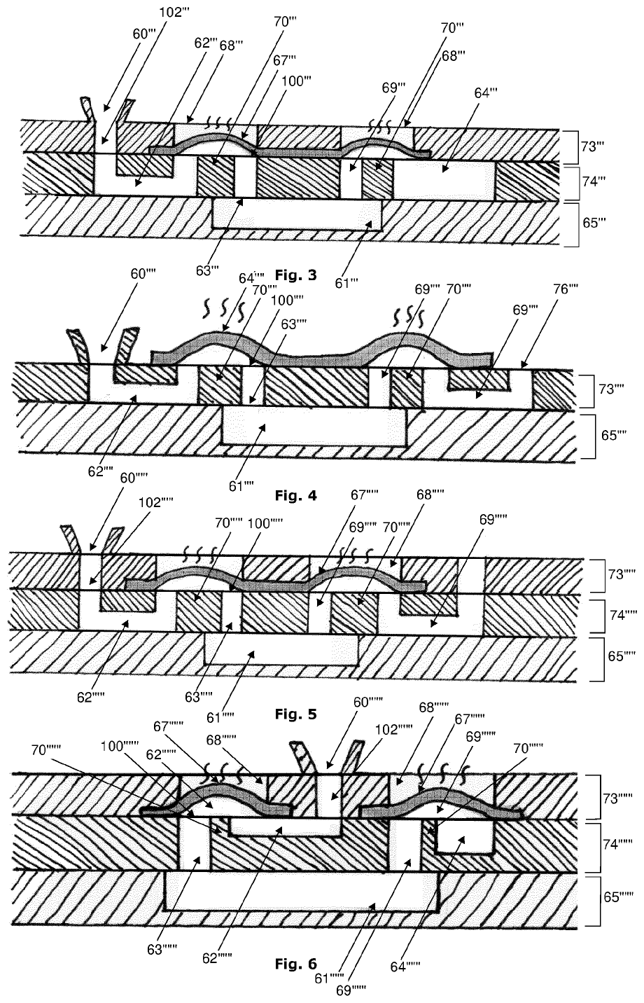

To better exemplify the possible different embodiments of a fluidic device in accordance with the present invention reference is made to FIGS. 1A, 1B, 2, 3, 4, 5 and 6. FIGS. 1A and 1B show respectively a top and a perspective view of an example of said device. FIG. 2 shows a perspective view of another example of said device. FIGS. 3, 4, 5 and 6 show a lateral cross section view of different examples of said device.

According to FIGS. 1A and 1B, the device comprises a top layer (73, 73′; 74, 74′), comprising an outer sub-layer (73, 73′), an inner sub-layer (74, 74′), and a bottom layer (64, 64′). The device comprises an injection opening (60, 60′), an inlet channel and a fluid distribution system in the outer sub-layer (73, 73′). The device further comprises multiple filling channels (63, 63′) and multiple waste wells (64, 64′) in the inner sub-layer (74, 74′). The device further comprises multiple light-transmissive wells (61, 61′) in the bottom layer. The fluid distribution system comprises bifurcating channels (62, 62′) comprising multiple distribution channels and multiple outlet ends (100, 100′). The fluid distribution system is in fluid connection with the filling channels (63, 63′) at the outlet ends (100, 100′). Each bifurcating channel (62, 62′) is in fluid connection with four filling channels (63, 63′) at the outlet ends (100, 100′). Each filling channel (63, 63′) is in fluid connection with one light-transmissive well (61, 61′).

When a fluid is inserted in the injection opening (60, 60′), the fluid will flow through the inlet channel into the fluid distribution system. The fluid will automatically be essentially equally distributed by the fluid distribution system over the light-transmissive wells (61, 61′) through the filling channels (63, 63′).

In theory it is possible to also construct a device whereby more than one filling channel (63, 63′) is in fluid connection with one light-transmissive well (61, 61′). Equal distribution of fluid over the present light-transmissive wells is important to make sure each well possesses enough fluid in order to conduct a meaningful experiment. The previous is especially important when dealing with wells comprising small volumes.

The fluidic device according to this example is also configured to simultaneously fill all the light-transmissive well with a fluid. In order to achieve the previous not all the bifurcating channels need to comprise the same length. By adding bends in the channel, the fluid can be made to flow slower through the channel. The fluid will be retained and would need more time to flow through the filling channel. The previous can also be achieved with varying the diameter of the different channels. In this example the outer bifurcating channels are longer but do not comprise as many bends as the inner channels. The simultaneous filling of light-transmissive is advantageous for experiments whereby time is essential, such as the determination of a minimum inhibitory concentration. Bifurcating channels are advantageous for obtaining the optimal device design. The dimensions of the device can hereby be minimized compared to the employment of non-bifurcating channels.

Each waste well is in fluid communication with a light-transmissive well (61, 61′) through a drainage channel (69, 69′). Waste wells are advantageous for limiting the messiness of the experiment. Moreover, they also give a good indication when a light-transmissive well is completely filled, ensuring that the light-transmissive wells (61, 61′) comprise enough interaction volume. Drainage channels are also advantageous for intercepting possible present volume differences in the filling of the light-transmissive wells. Also, contamination can hereby be minimized.

The other fluidic device example, FIG. 2, possesses similar elements as the fluidic device of FIG. 1A and 1B. However, this example comprises some key feature differences. Next to an injection opening (60″), an inlet channel and a fluid distribution system in the top layer (73″; 74″), comprising an outer sub-layer (73″) and an inner sub-layer (74″), multiple filling channels (63″) and multiple waste wells (64″) in the inner sub-layer (74″), and multiple light-transmissive wells (61″) in the bottom layer, whereby the fluid distribution system comprises bifurcating channels (62″) and multiple outlet ends (100″), the fluidic device according to FIG. 2 also comprises at least one porous membrane (67″) and vent openings (68″) present between the top (73″) and the inner sub-layer (74″). The vent openings are positioned in such a way that every filling channel (63″) is located underneath a vent opening (73″). The porous membrane is placed so that it is positioned between the filling channel (63″) and the vent openings (73″). Having a porous membrane in combination with vent openings is advantageous for the prevention or removal of air bubbles in the channels and/or light-transmissive wells. Especially when the light-transmissive wells are to be used in a photodetector system, the need to remove air bubbles is high. Further, the fluidic device comprises one waste well (64″) and a waste lead channel (75″). The waste lead channel is in fluid connection with all drainage channels (69″), and leads the fluid coming out of the drainage channels (69″) to the waste well (64″). Each bifurcating channel (62″) is also provided with a hump portion (70″) located underneath the vent opening and thus also the porous membrane. The hump portions are furthermore also provided in or near the outlet ends of the bifurcating channels.

The fluidic device examples in FIGS. 3, 4, 5 and 6 exemplify different possible fluidic device constructions.

The fluidic device of FIG. 3 comprises a top layer (73″′; 74″′), comprising an outer sub-layer (73″) and an inner sub-layer (74″), a bottom layer (65″′), an injection opening (60″′), an inlet channel (102″′), at least one bifurcating channel (62″′), at least one outlet end (100″′) of the bifurcation channel (62″′), a filling channel (63″′), a light-transmissive well (61″′), a waste well (64″′), a drainage channel (69″′), a porous membrane (67″′), and at least two vent openings (68″′). The injection opening, the inlet channel and the vent openings are located in the outer sub-layer. The porous membrane is located on top of the inner sub-layer. The bifurcating channel extends over the upper and inner top layers. The waste well is located in the inner sub-layer. The light-transmissive well is located in the bottom layer. In this example a part of each vent opening is a part of the bifurcating channel, whereby a part of the bifurcating channel wall is defined by the porous membrane. The fluid inserted through the injection opening will flow through the inlet channel, through the bifurcating channel, underneath the porous membrane (pushing due to the pressure build-up of the filling process the porous membrane to obtain a concave shape), through the filling channel to the light-transmissive well. Once the light-transmissive well is filled, the excess fluid will flow through the drainage channel, underneath the porous membrane, to the waste well. In this example the fluidic device comprises either one porous membrane or the same number of porous membranes as the number of light-transmissive wells.

The fluidic device of FIG. 4 comprises a top layer (73″″), a bottom layer (65″″), a injection opening (60″″), a bifurcating channel (62″″), an outlet end of the bifurcating channel (100″″), a filling channel (63″″), a light-transmissive well (61″″), a waste opening (76″″), a drainage channel (69″″) and a porous membrane (67″″). The injection opening, the filling channel and the drainage channel are located in the top layer. The porous membrane is located on top of the top layer. The drainage channel extends over the top layer and out of the top layer, underneath the porous membrane. The bifurcating channel extends over the top layer and out of the top layer, underneath the porous membrane (due to the pressure build-up of the filling process the porous membrane obtains a concave shape). The light-transmissive well is located in the bottom layer. The fluid inserted through the injection opening will flow through the bifurcating channel, underneath the porous membrane, through the filling channel to the light-transmissive well. Once the light-transmissive well is filled the excess fluid will flow through the drainage channel, underneath the porous membrane, to the waste opening where it will exit the fluidic device. In this example the fluidic device comprises either one porous membrane or the same number of porous membranes as the number of light-transmissive wells.

The fluidic device of FIG. 5 comprises a top layer (73″″′; 74″″′), comprising an outer sub-layer (73″″) and an inner sub-layer (74″″), a bottom layer (65″″′), an injection opening (60″″′), an inlet channel (102″″′), at least one bifurcating channel (62″″′), at least one outlet end (100″″′) of the bifurcation channel (62″″′), a filling channel (63″″′), a light-transmissive well (61″″′), a waste opening (76″″′), a drainage channel (69″″′), a porous membrane (67″″′), and at least two vent openings (68″″′). The injection opening and the vent openings are located in the outer sub-lay layer. The porous membrane is located on top of the top layer. The bifurcating channel extends over the inner sub-layer and the outer sub-layer. The waste opening is located in the inner sub-layer. The light-transmissive well is located in the bottom layer. In this example a part of each vent opening is a part of the bifurcating channel. The fluid inserted through the injection opening will flow through the inlet channel, through the bifurcating channel, underneath the porous membrane, through the filling channel to the light-transmissive well. Once the light-transmissive well is filled, the excess fluid will flow through the drainage channel, underneath the porous membrane, to the waste opening.

The fluidic device of FIG. 6 comprises a top layer (73″″′′; 74″″′′), comprising an outer sub-layer (73″″′′) and an inner sub-layer (74″″′′), a bottom layer (65″″′′), an injection opening (60″″′′), an inlet channel (102″″′′), at least one bifurcating channel (62″″′′), at least one outlet end (100″″′′) of the bifurcation channel (62″″′′), a filling channel (63″″′′), a light-transmissive well (61″″′′), a waste well (64″″′′), a drainage channel (69″″′′), at least two porous membranes (67″″′′), and at least two vent openings (68″″′′).The injection opening, the inlet channel and the vent openings are located in the outer sub-layer. The porous membranes are located on top of the inner sub-layer. The bifurcating channel extends over the inner sub-layer and the outer sub-layer. The waste well is located in the inner sub-layer. The light-transmissive well is located in the bottom layer. In this example a part of each vent opening is a part of the bifurcating channel. The fluid inserted through the injection opening will flow through the inlet channel, through the bifurcating channel, underneath a porous membrane, through the filling channel to the light-transmissive well. Once the light-transmissive well is filled, the excess fluid will flow through the drainage channel, underneath a porous membrane, to the waste well.

Each of the fluidic devices of FIGS. 3 to 6 furthermore also have a hump portion provided in each bifurcating channel and located underneath a vent opening and the porous membrane. The hump portions are furthermore also provided in or near the outlet ends of the bifurcating channels.

### Example 2: Definition of the Fluidic Device Specifications

During design of the fluidic device, the following aspects were tackled:
Dimension of each well and distance between 2 successive wells/channels
Device material
Optical quality of the different walls of the wells/channels

The optimal dimensions chosen for the selected laser sources for one well are 10 mm (length of the well), 1.5 mm (width of the well) and 1.5 mm (height of the well).

In order to eliminate cross talking (no light going from one channel to a neighboring channel) between the different wells and taking the dimensions of the source and detector used into account the optimal distance is 3.4 mm.

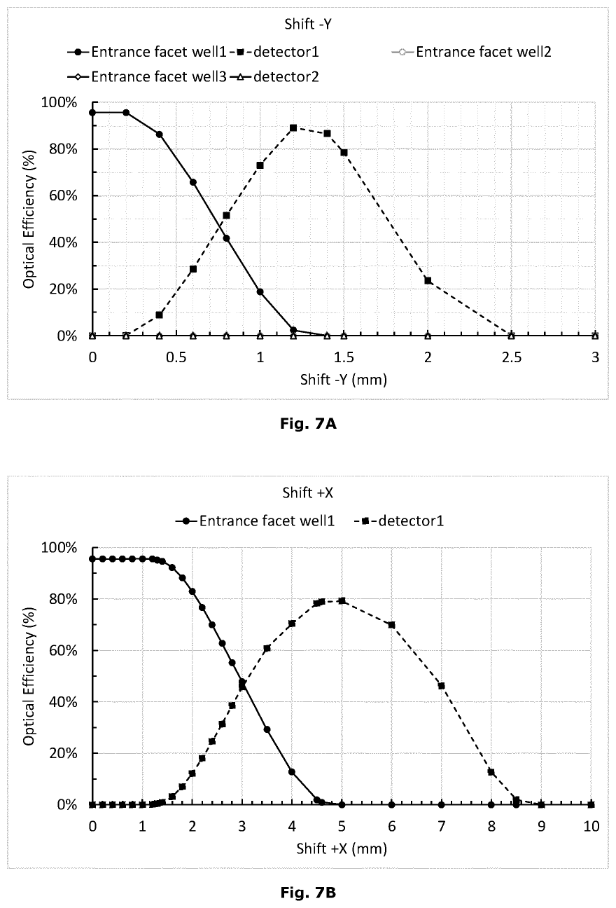

This is the case when applying the illumination first and second settings described below. The dimensions were selected based on the following considerations. The dimensions are matched to the dimensions of the illumination beam. The spot size is a little bit smaller than the channel cross section allowing some (FIGS. 7A and 7B) misalignment tolerances. The dimensions are large enough such that the required interaction volume is guaranteed. The entrance as disclosed in FIGS. 7A and 7B, should be understood as the portion of the well, wherein a light beam enters said well during for example optical analysis. The shift (X and Y) as disclosed in FIGS. 7A and 7B, should be understood as a shift of the light beam from an initial lighting configuration of a well in the horizontal (X) direction or the vertical (Y) direction with respect to the entrance of the initial lighting portion of said well.

‘First’ settings (laser beam directly out of laser):
Illumination spot shape: elliptical
Illumination spot diameter: 3.2 mm by 1 mm
Calculated dimension of illumination spot: (pi*3.2 mm*1 mm)/4=2.51 mm²
Calculated interaction volume: 2.51 mm²*10 mm (OPL)=25.1 mm³
Measured power at sample position: 3.97 mW
Calculated illumination power density: 3.97 mW/25.1 mm³=0.16 mW/mm³

‘Second’ settings (laser beam after cylindrical lenses):
Illumination spot shape: circular
Illumination spot diameter: 1 mm
Calculated dimension of illumination spot: (pi*1 mm*1 mm)/4=0.785 mm²
Calculated interaction volume: 0.785 mm²*10 mm (OPL)=7.85 mm³
Measured power at sample position: 3.97 mW
Calculated illumination power density: 3.97 mW/7.85 mm³=0.5 mW/mm³

The angle of incidence of the illumination beam determines which facets needs to be of an adequate optical surface quality. In this regard, the inventors note that an optical surface quality is adequate, as long as an RMS surface roughness is equal to at most a A used during optical analysis of the device's content divided by ten. In case of a collimated illumination beam, the light does not interact with the channel walls that therefore do not need an adequate optical quality. In the alternative case where the incoming light beam makes an angle with the entrance facet, an adequate optical quality of the channel walls and/or a reflective coating of said walls might also be required. The latter can for example be envisioned to increase the optical path length without increasing the sample volume.

The present invention is in no way limited to the embodiments described in the examples and/or shown in the figures. On the contrary, methods according to the present invention may be realized in many different ways without departing from the scope of the invention.

1. A multi-layer microfluidic device for determining a property of a microbe, comprising:
a bottom layer comprising a plurality of light-transmissive wells; and
a top layer comprising an injection opening and a fluid distribution system, wherein said fluid distribution system comprises a plurality of distribution channels, wherein said injection opening is in fluid connection to each of said plurality of distribution channels at an inlet end of each of said plurality of distribution channels and wherein each of said plurality of distribution channels is in fluid connection to one of said plurality of light-transmissive wells at an outlet end of each of said plurality of distribution channels;
characterized in that each of said plurality of distribution channels has:
essentially the same channel length between the inlet end and the outlet end; and
essentially the same channel volume.

2. The microfluidic device according to claim 1, wherein the fluidic device is configured to simultaneously fill each of the light-transmissive wells with a fluid inserted in the injection opening.

3. The microfluidic device according to claim 1, wherein each of the plurality of distribution channels is connected to a single injection opening.

4. The microfluidic device according to claim 1, wherein a path length between the injection opening and the outlet end of the distribution channel is essentially equal for each of the light-transmissive wells.

5. The microfluidic device according to claim 1, wherein said device comprises a waste drainage system and a waste well, wherein said waste drainage system comprises a plurality of drainage channels, wherein said waste well is provided in the top layer of said device, wherein each of said plurality of drainage channels is in fluid connection to one of said plurality of light-transmissive wells at an inlet end of each of said plurality of drainage channels and wherein each of said plurality of drainage channels is in fluid connection to said waste well at an outlet end of each of said plurality of drainage channels.

6. The microfluidic device according to claim 5, wherein each of said plurality of distribution channels and/or each of said plurality of drainage channels comprises an inlet portion at the inlet end of each channel, an outlet portion at the outlet end of each channel and a transportation portion connecting said inlet portion and said outlet portion, wherein said transportation portion is substantially provided along an outer surface of said top layer and/or along an outer surface of a sub-layer comprised by said top layer and wherein said outer surface is connected to an adjacent sub-layer or said bottom layer.

7. The microfluidic device according to claim 1, wherein the cross-sectional area of each of said plurality of distribution channels decreases near the outlet end.

8. The microfluidic device according to claim 5, wherein the device comprises a semi-permeable membrane disposed over at least a portion of a top side of each of said plurality of distribution channels and/or at least a portion of a top side of said plurality of drainage channels and/or at least a portion of a top side of said waste well, and wherein at least a portion of an outer surface of said membrane is provided along an outer surface of said microfluidic device.

9. The microfluidic device according to claim 8, wherein said top layer comprises an outer sub-layer and inner sub-layer, wherein said membrane is disposed between said outer sub-layer and said inner sub-layer and wherein said outer sub-layer is provided with a plurality of vent openings disposed over said semi-permeable membrane.

10. The microfluidic device according to claim 8, wherein said semi-permeable membrane is disposed over the top layer of said microfluidic device.

11. The microfluidic device according to claim 8, wherein a transportation portion of each of said plurality of distribution channels and/or each of said plurality of drainage channels comprises a hump portion extending along a width of each channel and wherein said semi-permeable membrane is substantially disposed over said hump portion.

12. The microfluidic device according to claim 11, wherein said semi-permeable membrane is adaptable between a flat resting state and a convex filling state.

13. The microfluidic device according to claim 12, wherein said semi-permeable membrane touches said hump portion when said semi-permeable membrane is provided in the flat resting state.

14. The microfluidic device according to claim 8, wherein said semi-permeable membrane is substantially disposed over an outlet portion of each of said plurality of distribution channels and/or each of said plurality of drainage channels.

15. The microfluidic device according to claim 1, wherein a fitting for an injection tool is provided at and/or in said injection opening.

16. The microfluidic device according to claim 1, wherein each of said plurality of light-transmissive wells comprises a first wall portion and a second wall portion, wherein the first and second wall portion are provided in a parallel configuration and wherein the first and second wall portion have an RMS surface roughness of at most 160 nm.

17. The microfluidic device according to claim 1, wherein each of said plurality of light-transmissive wells comprises at least one antimicrobial drug.

18. Use of the microfluidic device according to claim 1, for determining a susceptibility of a microbe to an antimicrobial drug.

19. The microfluidic device according to claim 17, wherein two or more of said plurality of light-transmissive wells comprise a substantially different concentration of said antimicrobial drug.

20. The use of the microfluidic device according to claim 18, wherein the microbe is a bacterium.
